(19) United States
(12) Patent Application Publication
(10) Pub. No.: US 2012/0123416 A1
(43) Pub. Date: May 17, 2012

(54) METHODS AND DEVICES FOR SOFT TISSUE AND JOINT REPAIR

(76) Inventors: Jeffrey GELFAND, Severna Park, MD (US); Robert S. Collins, Severna Park, MD (US); Hanspeter Robert Bayer, Meriden, CT (US); Ernest Corrao, JR., Bethel, CT (US)

(21) Appl. No.: 13/295,921

(22) Filed: Nov. 14, 2011

Related U.S. Application Data
(60) Provisional application No. 61/413,269, filed on Nov. 12, 2010.

(51) Int. Cl. A61B 17/16; A61B 17/10

(52) U.S. Cl. 606/79; 606/139

(57) ABSTRACT

Disclosed herein are methods and devices for tissue and joint repair. Devices as disclosed herein can be used during surgical procedures, for example, to repair biceps tendons, elbows, shoulders, and knees. Methods disclosed herein relate to methods for repairing the same using the disclosed devices.

### CROSS-REFERENCE TO RELATED APPLICATION

This application claims priority to U.S. Provisional Application for Patent Ser. No. 61/413,269 filed Nov. 12, 2010, the disclosure of which is incorporated herein by reference in its entirety.

### FIELD OF THE INVENTION

Disclosed herein are methods and devices for tissue and joint repair. Devices as disclosed herein can be used during surgical procedures, for example, to repair soft tissue and joints such as tendons, ligaments, elbows, shoulders, and knees. Methods disclosed herein relate to methods for repairing the same using the disclosed devices.

### BACKGROUND OF INVENTION

Soft tissue and joint repair procedures often involve the reattachment of soft tissue, such as tendons and ligaments, to bone. Currently available products for performing these procedures have various problems associated with their use. In one product, a button is secured to the soft tissue. Determining the length of the suture running from the soft tissue to the securing button is difficult. This length of suture cannot be changed so a surgeon may need to figure out/estimate the length, and presume that the soft tissue is properly secured to the bone. When a procedure is completed in this manner, there can be over-tension or slack in the system. These problems and others are overcome by the systems described herein.

### SUMMARY OF THE INVENTION

Accordingly, the present disclosure is directed to improvements in surgical repair, particularly, repair of soft tissue and joints, for example, distal biceps tendons, elbow ligaments, shoulders, and knees. In one embodiment, a repair system with an integrated means for imparting tension comprising a shuttle body having proximal end, a distal end, and a shaft connecting the proximal end and the distal end; an inserter slidably disposed in the shaft; a sleeve member disposed at the distal end of the shuttle body; and a means for imparting tension to a flexible material disposed at the proximal end of the shuttle body. The flexible material can be a suture relay for a surgical suture woven into a soft tissue graft. The system can include a tension securing means. In some embodiments, the sleeve can have a flange and a threaded internal bore for passage of the flexible material and reception of a tension securing means. The tension securing means is designed to work in cooperation with the sleeve.

In another embodiment, a repair system and associated method is adapted for reattaching the distal biceps tendon to the radial tuberosity. In accordance with this embodiment, the system includes a sleeve having a flange, a tension securing means, and a flexible material, for example, a suture. The system can also include means for imparting tension to the flexible material. In some embodiments, the sleeve defines a threaded internal bore for passage of the flexible material and reception of a tension securing means. The tension securing means is designed to work in cooperation with the sleeve.

### BRIEF DESCRIPTION OF THE DRAWINGS

FIG. 1 is a perspective view of a device as disclosed herein.

FIG. 2 is an exploded view of a device as disclosed herein.

FIG. 3 is a perspective view of a device as disclosed herein.

FIG. 4 is a perspective view of tension securing means as disclosed herein.

FIG. 5 is a perspective view of an adapter sleeve as disclosed herein.

FIG. 6 is a perspective view of a toggle as disclosed herein.

FIG. 7 is a perspective view of a toggle as disclosed herein disposed on an inserter.

FIG. 8 is a schematic representation of soft tissue being reattached to bone using a method and device as disclosed herein.

FIG. 9 is a schematic representation of a device as disclosed herein being used in a knee repair procedure.

FIG. 10 is a schematic representation of a biceps tendon being reattached to the radius using a method and device as disclosed herein.

### DETAILED DESCRIPTION

The repair system of the present disclosure can be used in surgical procedures and has particular application in securing soft tissue, including tendons, ligaments, etc., to bone. The system can be used in conjunction with surgery performed on the elbow, shoulder, back, ankle, knee, etc. and has particular application in biceps repair, elbow repair, shoulder repair, knee repair, foot repair, and ankle repair. Such biceps repair operations include, but are not limited to, reattachment of a torn or damaged biceps tendon, for example distal biceps tendon repair. Such elbow repair operations include, but are not limited to, repair of a torn or damaged elbow ligament, for example, by ulnar collateral ligament (UCL) surgery. Such shoulder repair operations include, but are not limited to, repair of a torn or damaged shoulder ligament, for example, by acromioclavicular (AC) surgery. Other procedures are also envisioned.

A repair system as disclosed herein can include, for example, at least three components, in particular, a sleeve for mounting to a side of a bone and/or positioning within a bone tunnel, a tension securing means for securing tension on the flexible material, and a flexible material (e.g., a suture).

Suitable tension securing means include a set screw. For example, a set screw as described in United States published patent application number US 2010/0262185, which is incorporated herein in its entirety.

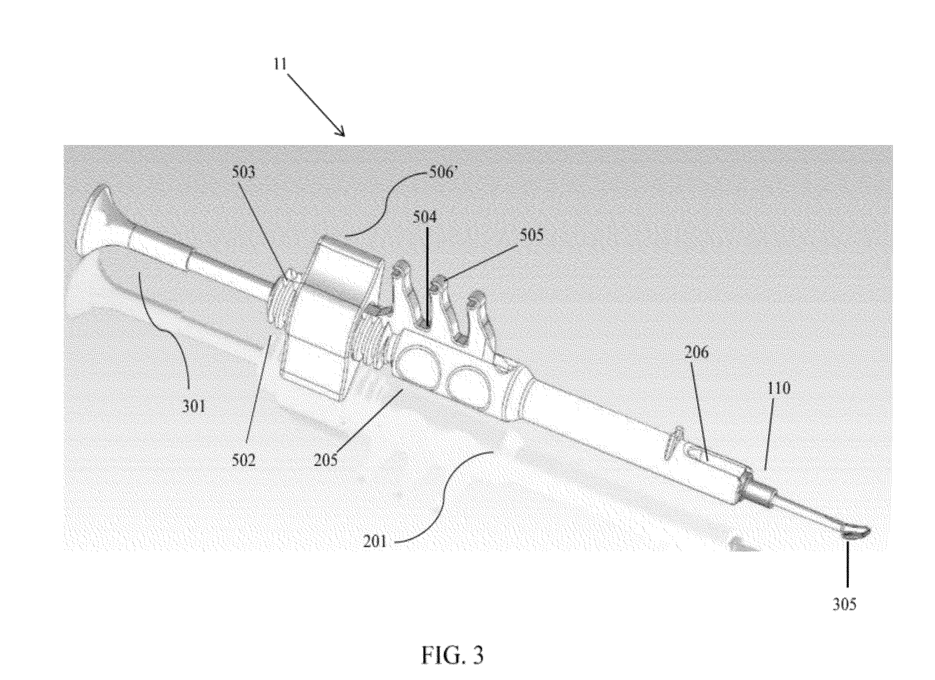

Referring to FIG. 3, another suitable tension securing means is an insertion piece 800 having a spherical portion 801 integrally formed with a base portion 802, for example, which can have a convex face 803. The insertion piece can have a shaft 804 for passing a flexible material, such as a suture, therethrough. The shaft can be positioned where the spherical portion and base portion meet. The sleeve of the repair system can be adapted for use with the insertion piece, for example, by having a flange and a smooth internal bore for reception of the spherical portion, in addition to passage of the flexible material. The internal bore of the sleeve can decrease in diameter away from the flange so that the spherical portion can be inserted within the sleeve such that the edge of the base portion is flush with the top of the internal bore or slightly inside the internal bore. The sleeve can also have a shaft connecting the internal bore to the edge of the flange for passage of the flexible material. In other words, a flexible material can pass through the insertion piece and sleeve.

The flexible material can pass through the shafts in the insertion piece and the flange of the sleeve such that application of tension to the flexible material urges the insertion piece into the sleeve. This provides a self-energizing mechanism in which securing strength builds up as more tension is applied to the flexible material. The securing means can further comprise a removable rod to act as a cantilever spring to exert pressure on the insertion piece to bias it into locking engagement with the sleeve and to prevent lateral slipping of the insertion piece upon initial tensioning of the flexible material.

The repair system described herein can further comprise means for imparting tension to the flexible material (not shown). One suitable means for imparting tension on a flexible material is described in U.S. patent application Ser. No. 12/944,379 filed Nov. 11, 2010, the entire contents of which are specifically incorporated herein by reference.

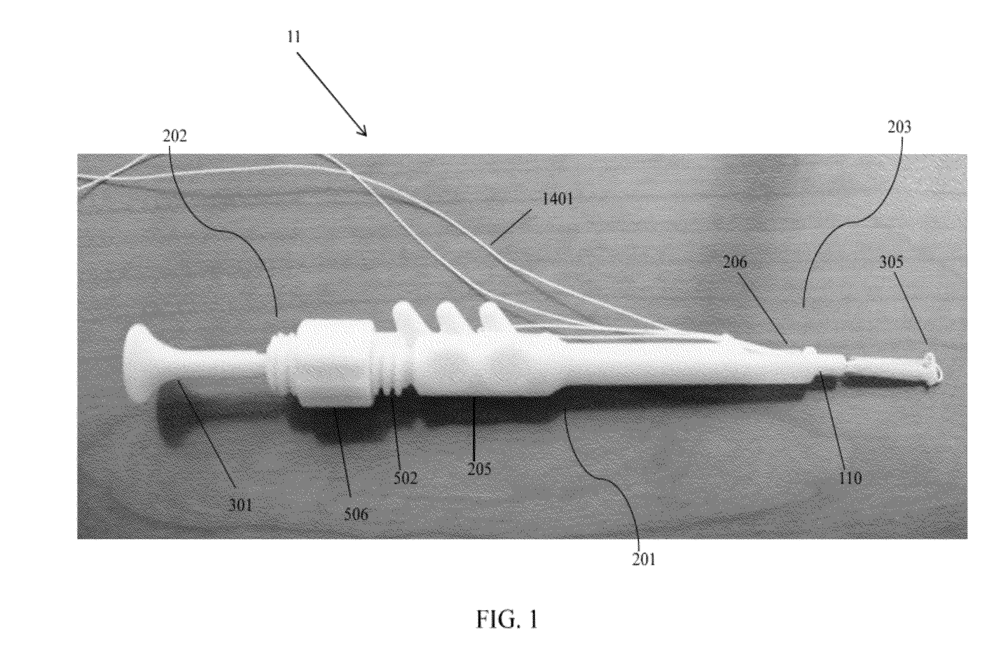

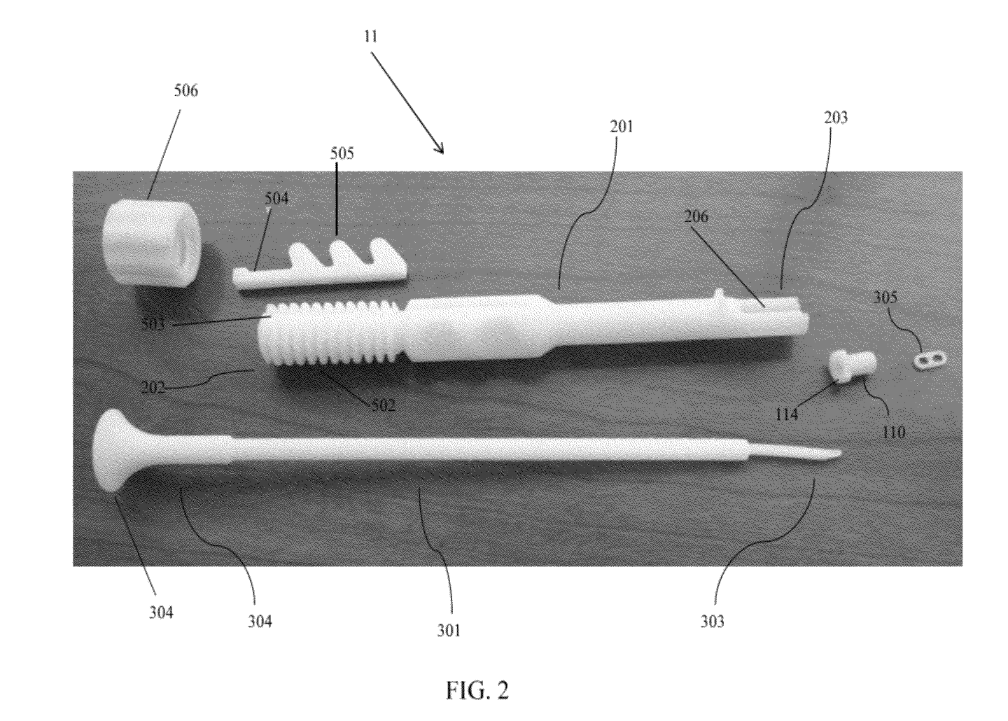

As another suitable means for imparting tension, the repair system can further comprise at least one anchoring means for anchoring the flexible material, for example a hook or cleat. The hook or cleat can have a base. A plurality of hooks or cleats can be integrally formed on a single base. The means for imparting tension can further comprise a threaded piece having a channel in which a base portion of the at least one hook is slidably disposed. A tension adjustment knob can be placed around the threaded piece and over at least a portion of the base of the cleat. The tension adjustment knob can be a knob as shown in FIGS. 1 and 2 or a wing nut-type knob as shown in FIG. 3. Rotation of the tension adjustment knob can slide the base and cleat within the channel thereby adjusting the tension of the flexible material anchored to the cleat. The cleat can be constructed to bias towards the distal end of the shuttle body as tension on the anchored flexible material is increased. For example, as tension on the flexible material is increased, the cleat can bend towards the distal end of the shuttle body and, as it approaches becoming perpendicular to the base portion, can be an indication that proper tension is imparted to the flexible material. The means for imparting tension can be integrated with the repair system.

In another embodiment, a repair system disclosed herein can be adapted for use in surgical procedures to repair joints, for example, elbows, shoulders, and knees. This repair system can have an integrated means for imparting tension to a flexible material (e.g., suture means).

Referring to FIGS. 1 to 3, the repair system 11 can comprise a shuttle body 201 having a proximal end 202, a distal end 203, and a shaft connecting the proximal end and the distal end and which can provide external communication therethrough; an inserter 301 slidably disposed in the shaft; a sleeve member 110 disposed at the distal end of the shuttle body; and means for imparting tension to a flexible material integrated with the shuttle body, for example at the proximal end of the shuttle body. The repair system can further comprise tension securing means, as described above. As used with reference to FIGS. 1 to 3, “proximal end” refers to the end of the shuttle body near the operator of the repair system and “distal end” refers to the end of the shuttle body away from the operator of the repair system.

The shuttle body can have any cross-section, for example, it can have a square cross-section. Other cross-sections can include, but are not limited to, circular, oval, and polygonal (e.g., hexagonal) cross-sections. The shuttle body can be integrated with the means for imparting tension. For example, the proximal end of the shuttle body can be a threaded portion 502 of a means for imparting tension. The threaded portion 502 can define a channel 503 in which a base 504 of at least one cleat 505, e.g., a hook, is slidably disposed. A tension adjustment knob 506 (FIG. 1 and 2) or 506′ (FIG. 3) can be threadedly attached to the threaded portion and over the base in order to slide the base and hook within the channel to adjust the tension of the flexible material anchored to the hook. The shuttle body can further comprise a grip section 205 on the outside of the body between the proximal end and the distal end to increase the ease of handling the repair system by an operator. The channel defined by the threaded portion can extend into the grip section thereby increasing the distance range for movement of the base and cleat.

The shaft of the shuttle body can have any cross-section, for example, it can have a square cross-section and is disposed in the housing. Other cross-sections can include, but are not limited to, circular, oval, and polygonal (e.g., hexagonal) cross-sections.

The distal end of the shuttle body can be adapted to hold the sleeve member in place. Additionally, the distal end of the shuttle body can have an opening 206 for passage of a flexible material.

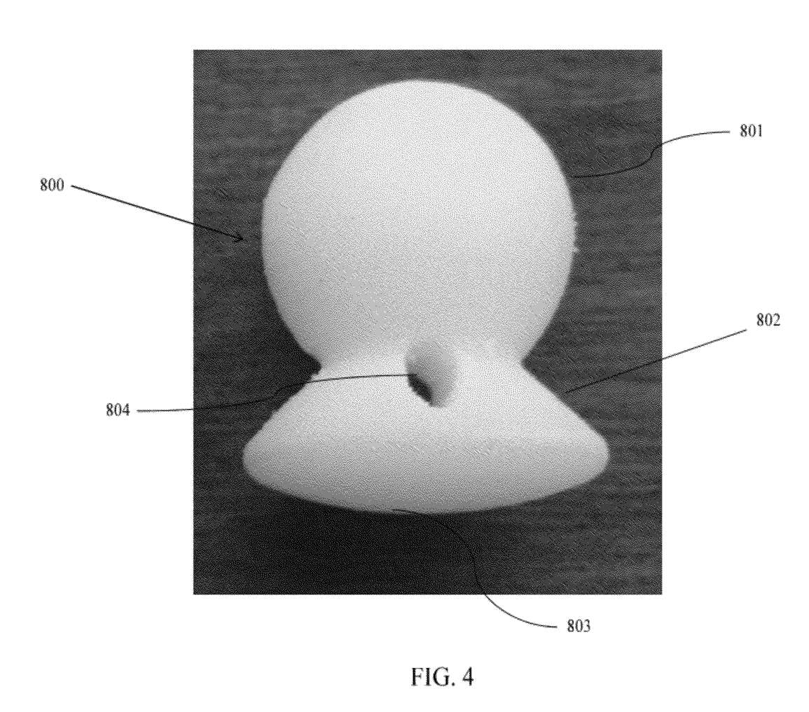

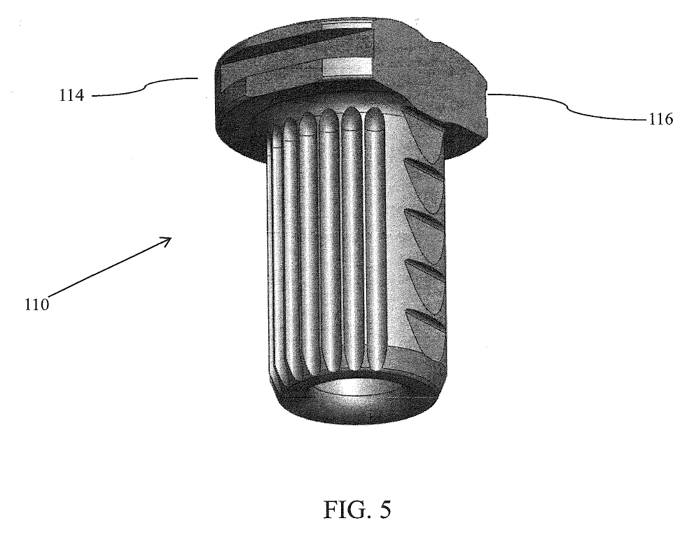

The sleeve member 110 can have a flange 114, which may be formed as part of the sleeve or may be a separate piece (e.g., a washer), and which is shaped for insertion into the distal end of the shuttle body. As shown in FIG. 4, the flange 114 can be generally circular with at least one flat side 116. The sleeve member can have an internal bore, for example a threaded or non-threaded bore, for passage of a flexible material and distal portion of the inserter therethrough. The sleeve member can be configured based on a particular means for imparting tension and tension securing means. For example, when using a set screw as means for securing tension, the sleeve member can have a threaded internal bore. When using the above-described insertion piece as means for securing tension, the sleeve member can have a smooth internal bore.

The inserter 301 can have a proximal end 302 and a distal end 303 and can be, for example, a toggle inserter. The shape and dimensions of the inserter are not particularly limited; however, the inserter should be formed so as to be able to slide through the shaft of the shuttle body from application of pressure by a user and to maintain its position within the shaft when the user is not applying pressure. The proximal end of the inserter can be shaped so as to prevent complete insertion of the inserter into the shaft of the shuttle body. As shown in FIG. 2, the inserter may be concave at its proximal end and may have a rim protrusion 304 which allows the user to easily apply pushing pressure for movement of the inserter through the shaft to the distal end of shuttle body and to pull the inserter for movement towards and removal through the proximal end of the shuttle body. As shown in FIG. 2, the inserter can comprise sections which decrease in diameter from the proximal end to the distal end. The distal-most end of the inserter can be adapted for placement of a toggle 305, for example, by being slightly angled in relation to the rest of the inserter to form a hook.

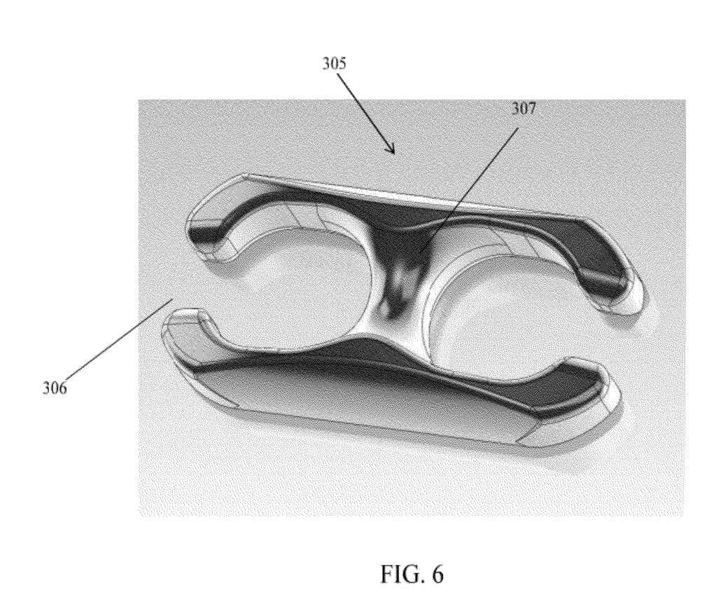

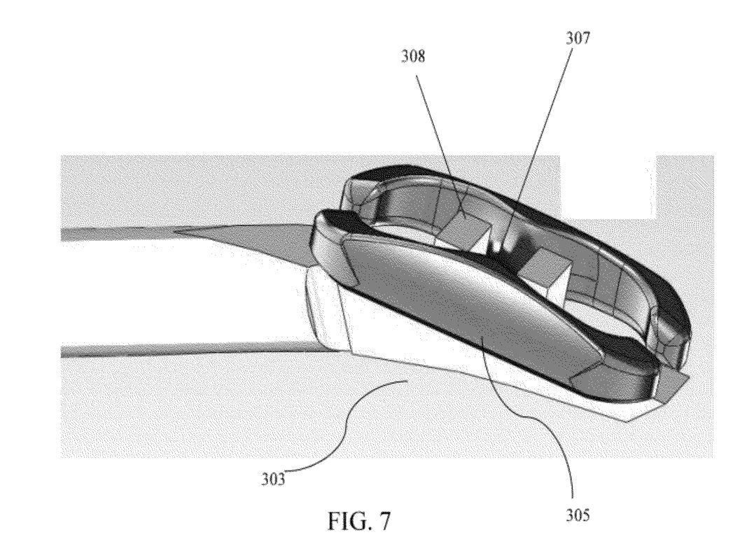

The toggle 305 can be provided with closed ends, as shown in FIG. 2. Alternatively, the toggle can be provided with partially open ends 306, as shown in FIG. 6. When provided with partially open ends, the toggle can be positioned on the inserter as shown FIG. 7. For example, the distal-most end of the inserter can be provided with protrusions 308 for receiving a connecting section 307 of the toggle. A repair system having the toggle as shown in FIG. 7 can operate without a shuttle suture. Such a configuration allows placement of a suture through soft tissue, for example, a tendon, before positioning the toggle on the bone of a patient. Thus, it is not necessary to pass the suture through the toggle which avoids passing the toggle through a hole to a side of a patient's bone which is not accessed.

In use, for example, in joint repair surgery, the repair system 11 can be assembled as shown in FIG. 1 or FIG. 3, with a shuttle body 201 having an integrated means for imparting tension to a flexible material 1401, i.e., a tension adjustment knob 506 and a threaded portion 502 defining a channel 503 in which a plurality of cleats 505 on a base 504 are slidably disposed. The sleeve member 110 is inserted in the distal end of the shuttle body. The inserter 301 is positioned through the shaft of the shuttle body and extending through the distal end of the shuttle body and through the internal bore of the sleeve member 110. A toggle 305 is positioned on the distal-most end of the inserter 301. A flexible material 1401 is threaded through the toggle and passes through the internal bore of the sleeve and the opening 206 and the distal end of the shuttle body.

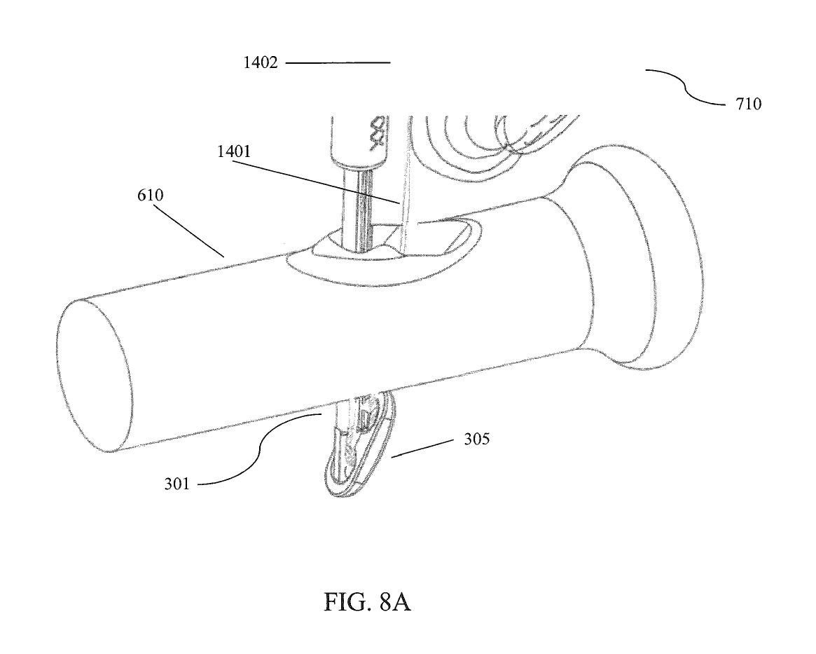

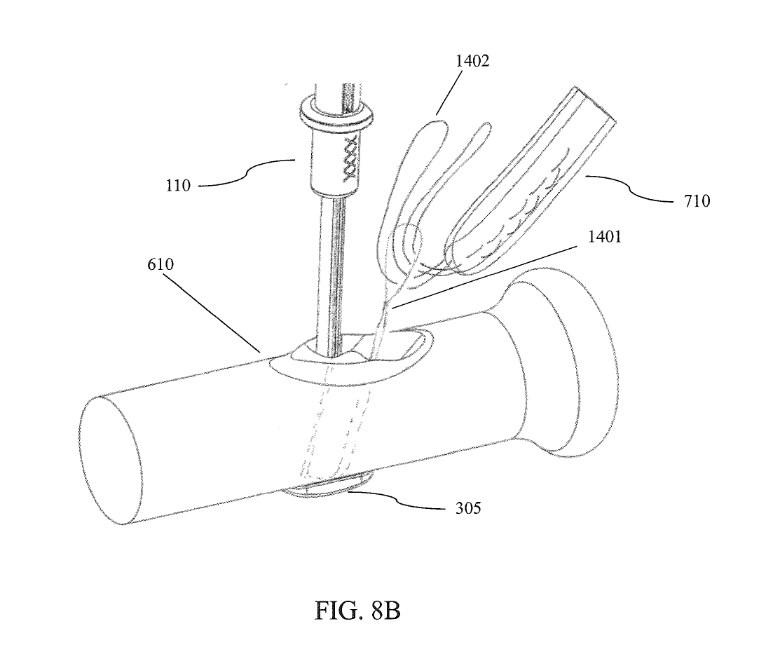

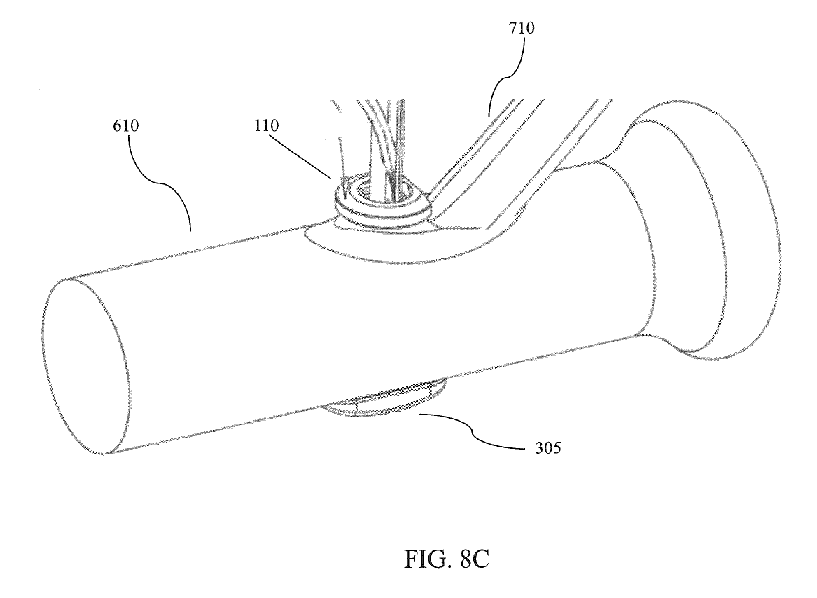

Referring to FIGS. 8A to 8C, a hole is drilled through a patient's bone 610. The inserter and toggle 305, with a flexible material 1401, for example, a suture relay, are inserted through the hole and the toggle is positioned onto one side of the hole. The toggle is detached from the inserter and the inserter is withdrawn through the hole. In order to facilitate detachment of the toggle, the hole can be drilled to have a diameter smaller than the length of the toggle. As the toggle passes through the hole, it is compressed. Upon passing to the other side of the drilled hole, the release of compression can cause the toggle to detach from the inserter. The inserter is then removed from the shuttle body. Removal of the inserter from the shuttle body permits insertion of additional components for use in repair procedures, for example, a torque driver.

An additional flexible material 1402, for example, a whip stitch suture which is attached at its opposite end to soft tissue 710, is attached to an end of the flexible material and pulled through an opening of or around a connecting section of the toggle 305 and then through the sleeve member 110 and the shuttle body opening and affixed to the cleat of the integrated means of imparting tension (not shown). The sleeve member 110 is then positioned on the side of the drilled hole opposite the toggle 305 and the additional flexible material is tensioned by rotating the tension adjustment knob (not shown). A tension securing means (not shown) is inserted into the sleeve to maintain the tension generated by the means for imparting tension. The additional flexible material is removed from the cleat and shuttle body is detached from the sleeve.

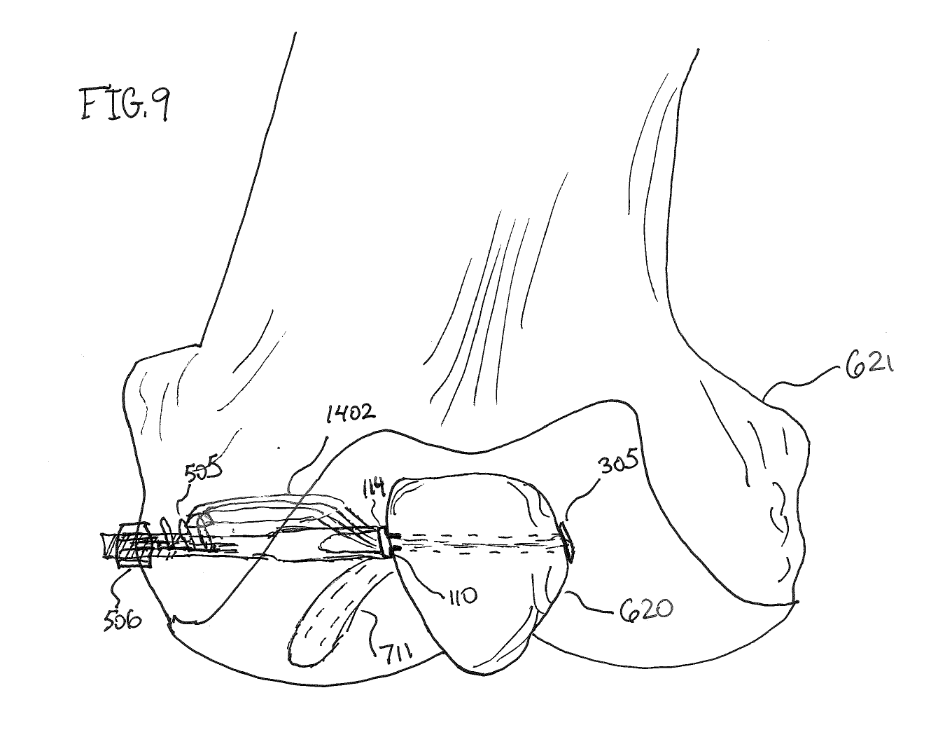

FIG. 9 illustrates a device disclosed herein positioned for use in surgical repair of a knee, particularly for reattaching soft tissue 711 to the patella 620 (show in front of femur 621). Toggle 305 is positioned on one end of a hole drilled through the patella using an inserter (not shown). In order to facilitate detachment of the toggle, the hole can be drilled to have a diameter smaller than the length of the toggle. As the toggle passes through the hole, it is compressed. Upon passing to the other side of the drilled hole, the release of compression can cause the toggle to detach from the inserter. The inserter is then removed from the shuttle body. Removal of the inserter from the shuttle body permits insertion of additional components for use in repair procedures, for example, a torque driver.

A flexible material 1402, for example, a whip stitch suture which is attached at its opposite end to soft tissue 711, is attached to an end of the flexible material and pulled through an opening of or around a connecting section of the toggle 305 and then through the sleeve member 110 and the shuttle body opening and affixed to the cleat 505 of the integrated means of imparting tension. The sleeve member 110 is then positioned on the side of the drilled hole opposite the toggle 305 and the additional flexible material is tensioned by rotating the tension adjustment knob 506. A tension securing means (not shown) is inserted into the sleeve to maintain the tension generated by the means for imparting tension. The additional flexible material is removed from the cleat and shuttle body is detached from the sleeve.

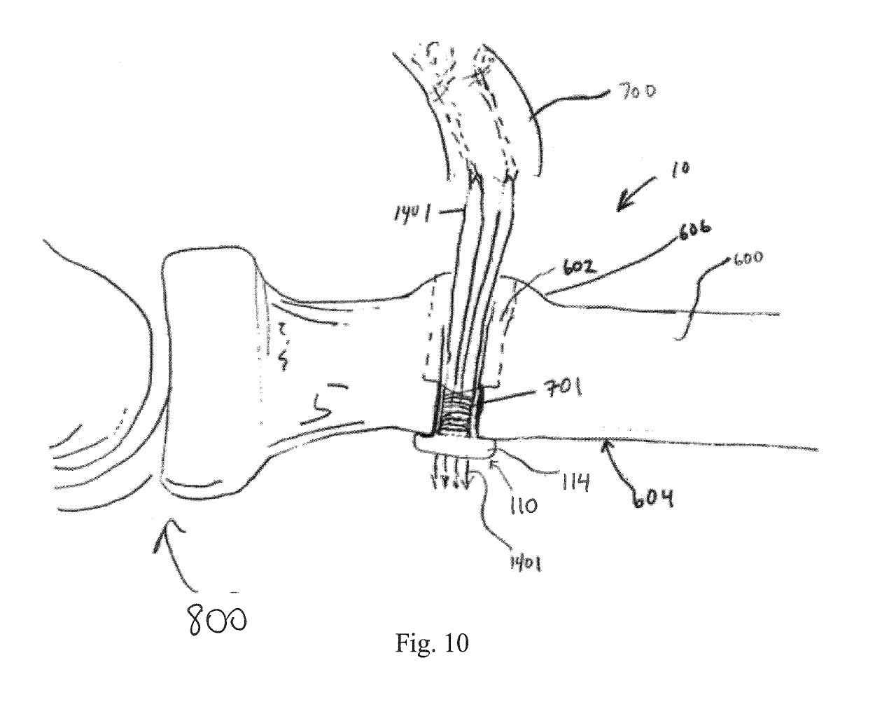

FIG. 10 illustrates another repair system as disclosed herein. Repair system 10 generally includes three components, in particular, a sleeve 110 for mounting to a side of the bone, tension securing means 701 for securing tension on the flexible material, and a flexible material 1401 (e.g., suture means).

In use, for example in a distal biceps tendon repair surgery, sleeve 110 rests on a posterior aspect of the proximal radius 604 just opposite to the radial tuberosity 606. The sleeve 110 can have a flange 114 which may be formed as part of the sleeve or may be a separate piece (e.g., a washer). The flange 114 supports the sleeve on the posterior aspect of the proximal radial cortex while the body of the sleeve passes partially through a drilled hole 602 connecting the posterior and anterior surfaces of the proximal radius at a level of a normal, intact biceps insertion. The sleeve 110 may include a passage for a flexible material 1401, (e.g. suture, tape, or other suitable material) that is attached on its proximal end to an avulsed, injured, biceps tendon 700. The free ends of the flexible material are passed through the sleeve, tensioned appropriately and secured by inserting tension securing means (not shown), for example, the tension securing means as described in United States published patent application number US 2010/0262185, in the sleeve.

In one embodiment the flexible material 1401 is secured to the injured, avulsed biceps tendon 700 through an anterior incision about the elbow 800. The radial tuberosity 606 is exposed and a small gauge pin or drill which has an eyelet for receiving free ends of said flexible material (suture to be placed through eyelet later in procedure) is advanced through the proximal radius 604 from anterior to posterior at the site of the normal insertion of the biceps tendon 700. The pin may then be advanced out the posterior aspect of the forearm. The flexible material 1401 is then threaded through the eyelet in the pin. In one embodiment a cannulated device may be placed over the pin as it protrudes out the posterior aspect of the forearm and advanced over the wire so that it rests on the posterior aspect of the radius. This cannulated device is designed to protect the surrounding soft tissues during the remainder of the procedure. A cannulated drill may then be passed over the previously placed pin and drilled through the posterior cortex. The sleeve may then be passed down cannulated device to rest on posterior cortex-as previously mentioned. The pin can then be pulled out posteriorly with the flexible material threaded through its trailing end so that when the pin is removed free ends of flexible material now protrude out of the cannulated device. A tensioner may be mounted on cannula or may engage threaded sleeve 110 through an engagement member, tension can then be applied to flexible material 1401 so that biceps tendon 700 is brought into anterior hole 602 that will be created in the radial tuberosity through an earlier step. With appropriate tension maintained on flexible material, a tension securing means will be passed down cannula and inserted into sleeve using appropriate torque.

For the purposes of illustration and without limitation, the term “suture”, “suture means”, “flexible material” as used herein may be a cable, filament, thread, wire, fiber tape, or any other flexible member suitable for soft tissue and bone fixation in the body.

While the foregoing disclosure has been described in some detail for purposes of clarity and understanding, it will be appreciated by one skilled in the art from a reading of this disclosure that various changes in form and detail can be made without departing from the true scope of the disclosure and appended claims. All patents and publications cited herein are entirely incorporated herein by reference.

What is claimed is:

1. A method comprising:
accessing an internal portion of a bone area of a patient;
creating a hole through the bone of the patient;
mounting a sleeve member on one end of the hole;
positioning a toggle on an end of the hole opposite the sleeve member;
attaching a flexible material to the soft tissue and passing the flexible material through the sleeve member;
attaching the flexible material to a means for imparting tension;
tensioning the flexible material with the means for imparting tension; and
applying a tension securing means to the sleeve member.

2. The method according to claim 1, wherein the sleeve member comprises a flange and a threaded internal bore.

3. The method according to claim 1, wherein the sleeve member comprises a flange and a smooth internal bore.

4. The method according to claim 1, wherein said toggle is positioned with a toggle inserter.

5. The method of claim 1, wherein the diameter of the hole through the bone is smaller than the length of the toggle.

6. The method of claim 1, wherein the means for imparting tension is integrated with a device which mounts the sleeve member.

7. The method of claim 2, wherein the tension securing means comprises a set screw.

8. The method of claim 3, wherein the tension securing means comprises an insertion piece having a spherical portion integrated with a base portion, wherein the spherical portion is inserted into the internal bore of the sleeve member.

9. A device comprising:
a shuttle body having proximal end, a distal end, and a shaft connecting the proximal end and the distal end;
an inserter slidably disposed in the shaft;
a sleeve member disposed at the distal end of the shuttle body; and
an integrated means for imparting tension disposed on the shuttle body.

10. The device of claim 9, wherein the means for imparting tension is disposed on the proximal end of the shuttle body.

11. The device of claim 9, wherein the means for imparting tension comprises a tension adjustment knob, a cleat having a base portion, and a thread having a channel in which the cleat and base portion are slidably disposed.

12. The device of claim 11, wherein said cleat biases towards the distal end of the shuttle body when tension is imparted on a flexible material anchored on the cleat.

13. The device of claim 9, wherein the sleeve member comprises a flange and an internal bore.

14. The device of claim 9, further comprising a toggle positioned on a distal-most end of the inserter.

15. The device of claim 14, wherein the toggle is configured to compress when passing through a hole in a bone.

16. The device of claim 14, wherein the toggle has closed ends.

17. The device of claim 14, wherein the toggle has partially open ends.

18. The device of claim 9, further comprising a tension securing means.

19. The device of claim 18, wherein the sleeve member comprises a flange and a threaded internal bore.

20. The device of claim 18, wherein the sleeve member comprises a flange and a smooth internal bore.

21. The device of claim 19, wherein the tension securing means is a set screw.

22. The device of claim 20, wherein the tension securing means comprises an insertion piece having a spherical portion integrated with a base portion.

23. A method comprising:
accessing an internal portion of a bone area of a patient;
creating a hole through the bone of the patient;
mounting a sleeve member on one end of the hole;
attaching a flexible material to the soft tissue and passing the flexible material through the sleeve member;
attaching the flexible material to a means for imparting tension;
tensioning the flexible material with the means for imparting tension; and
applying a tension securing means to the sleeve member.

24. The method according to claim 23, wherein the sleeve member comprises a flange and a threaded internal bore.

25. The method according to claim 23, wherein the sleeve member comprises a flange and a smooth internal bore.

26. The method of claim 24, wherein the tension securing means comprises a set screw.

27. The method of claim 25, wherein the tension securing means comprises an insertion piece having a spherical portion integrated with a base portion, wherein the spherical portion is inserted into the internal bore of the sleeve member.
